(19) United States
(12) Patent Application Publication
(10) Pub. No.: US 2012/0183504 A1
(43) Pub. Date: Jul. 19, 2012

(54) COMPOSITION AND USE OF PROBIOTIC STRAIN GM-263 (ADR-1) IN TREATING RENAL FIBROSIS IN DIABETES

(76) Inventors: Ying-Chen LU, CHIAYI CITY (TW); Jau-Shyang HUANG, Tainan City (TW); Feng-Ching HSIEH, Pingtung City (TW); Yi-Cheng LI, KAOHSIUNG CITY (TW); Chia-Lin LEE, TAINAN CITY (TW)

(21) Appl. No.: 13/006,432

(22) Filed: Jan. 14, 2011

(51) Int. Cl. A61K 35/74; A61P 13/12; A61P 3/10

(52) U.S. Cl. 424/93.3; 424/93.45

(57) ABSTRACT

A use of probiotic strain GM-263 (ADR-1) in treating renal fibrosis in diabetes is disclosed. The probiotic strain such as Lactobacillus renteri strain GM-263 (ADR-1) (accession No. CCTCC M 209263) is utilized to produce a composition for treating renal fibrosis in diabetes in an effective dose, thereby reducing the concentration of glycated hemoglobin and blood sugar and keeping body weight and kidney weight within normal range, as well as specifically inhibiting phosphorylation of JAK2/STAT1 signal transduction pathway and renal fibrosis-related protein expression.

### FIELD OF THE INVENTION

This invention relates generally to a composition and use of probiotic strain, and more particularly, to a composition and use of probiotic strain GM-263 (ADR-1) for treating renal fibrosis in diabetes.

### BACKGROUND OF THE INVENTION

Hyperglycemia is known to be a major risk factor of diabetic nephropathy (DN) 40% of dialysis patients are diabetic patients having DN, and DN is the main cause of end-stage renal disease (ESRD); DN usually starts to appear clinical changes after diabetes has been present for 15 to 20 years. Very high percent of DN will develop ESRD. Other complications such as hypertension, hyperlipidemia, hyperuricemia and cardiovascular diseases may also occur.

DN is roughly divided into five following stages of hyperfiltration, silent phase, microalbuminuria, proteinuria and ESRD. Typically, DN can be treated by controlling blood glucose, blood pressure, diet, drugs and so on, so that the blood glucose, blood pressure and protein uptake can be controlled.

Generally, probiotics (or probiotic bacteria) such as lactic acid bacteria (LAB) and some yeasts, are referred to live microorganisms for beneficial to gastrointestinal (GI) tract health, which are supplements or originally inhabit in the human body for beneficial to gastrointestinal (GI) tract health. As such for LAB, they are named as such because most of their members convert lactose and other sugars into lactic acid. LAB is also a genus of Gram-positive facultative anaerobic or microaerophilic bacteria, and they are widely applied in fermentation of food industry.

Many researches evidence that LAB can improve the allergy-related diseases and GI upset, for example, inflammatory bowel disease (IBD). Moreover, LAB can stimulate the immune response, so as to promote the immune tolerance to innocent allergens. Besides, LAB have been evaluated in other research studies in animals and humans with respect to antibiotic-associated diarrhea, travellers' diarrhea, pediatric diarrhea, inflammatory bowel disease, irritable bowel syndrome, atopic disease and so on in other studies.

However, prior studies are little or irrelevant to whether the probiotics can prevent renal fibrosis in diabetes, so that they fail to discuss the potential mechanism of the probiotics for preventing renal fibrosis in diabetes.

Therefore, it is necessary to provide a new use of the probiotic strain for treating renal fibrosis in diabetes, thereby developing other applications of the probiotic strains.

### SUMMARY OF THE INVENTION

Accordingly, an aspect of the present invention provides a composition for treating renal fibrosis in diabetes is disclosed, which may comprise a therapeutically effective amount of a probiotic strain GM-263 (ADR-1) of Lactobacillus reuteri strain GM-263 (ADR-1). L. reuteri strain GM-263 (ADR-1) has been deposited with the China Center for Type Culture Collection (CCTCC), Wuhan University, Wuhan 430072, People's Republic of China under accession number of CCTCC M 209263 on Nov. 13, 2009. The probiotic strain GM-263 (ADR-1) can effectively reduce the concentration of glycated hemoglobin and blood sugar and keep body weight and kidney weight within normal range, thereby treating renal fibrosis in diabetes.

Another aspect of the present invention provides a method for treating renal fibrosis in diabetes. The method comprises to administrate a therapeutically effective amount of probiotic strain GM-263 (ADR-1) of Lactobacillus reuteri strain GM-263 (ADR-1) (accession No.: CCTCC M 209263), in which the probiotic strain GM-263 (ADR-1) can specifically inhibit phosphorylation of JAK2/STAT1 signal transduction pathway and renal fibrosis-related protein expression, thereby treating renal fibrosis in diabetes.

According to the aforementioned aspect of the present invention, a composition for treating renal fibrosis in diabetes is disclosed. In an embodiment, the composition may comprise a therapeutically effective amount of a probiotic strain GM-263 (ADR-1) of Lactobacillus reuteri strain GM-263 (ADR-1) (deposited with CCTCC of Wuhan University in China under accession No.: CCTCC M 209263).

In an embodiment, the probiotic strain GM-263 (ADR-1) may be live or inactivate.

In another embodiment, the probiotic strain GM-263 (ADR-1) specifically inhibits phosphorylation of JAK2/STAT1 signal transduction pathway and renal fibrosis-related protein expression. The renal fibrosis-related proteins are, for example, plasminogen activator inhibitor (PAI-1), cyclin-dependent kinase inhibitor (CDK1) P21^{Waf1/Cip1}, smooth muscle α-actin (α-SMA) or fibronectin.

In a further embodiment, the probiotic strain GM-263 (ADR-1) may be a medical composition, a food additive, a food or its ingredient.

According to other aspects of the present invention, a method for treating renal fibrosis in diabetes is disclosed. The method comprises administrating a therapeutically effective amount of probiotic strain GM-263 (ADR-1) of Lactobacillus reuteri strain GM-263 (ADR-1) (deposited with CCTCC of Wuhan University in China under accession No.: CCTCC M 209263) in which the probiotic strain GM-263 (ADR-1) specifically inhibits phosphorylation of JAK2 and STAT1, and also inhibits protein expression of PAI-1, CDKI P21^{Waf1/Cip1}, α-SMA or fibronectin.

With application to the aforementioned probiotic strain GM-263 (ADR-1) for treating renal fibrosis in diabetes, die probiotic strain GM-263 (ADR-1) can effectively reduce the concentration of glycated hemoglobin and blood sugar, and keep body weight and kidney weight within normal range. Moreover, the probiotic strain GM-263 (ADR-1) can treat renal fibrosis in diabetes by specifically inhibiting phosphorylation of JAK2/STAT1 signal transduction pathway and inhibiting renal fibrosis-related protein expression, thereby developing other applications of the probiotic strain GM-263 (ADR-1).

### BRIEF DESCRIPTION OF THE DRAWINGS

The foregoing aspects and many of the attendant advantages of this invention are more readily appreciated as the same become better understood by reference to the following detailed description, when taken in conjunction with the accompanying drawing, wherein:

FIGS. 1A and 1B show bar diagrams of L. reuteri GM-263 (ADR-1) treatment on glycated hemoglobin (FIG. 1A) and blood glucose (FIG. 1B) in rats according to an embodiment of the present invention.

FIG. 2 shows bar diagrams of L. reuteri GM-263 (ADR-1) treatment on body weight (FIG. 2A) and left kidney weight (FIG. 2B) of rats according to an embodiment of the present invention.

FIG. 3 shows a Western blotting analysis of renal cortex tissue of rats according to an embodiment of the present invention.

FIG. 4A depicts a bar diagram of the relative expression of JAK2 of FIG. 3 normalized to β-actin expression of FIG. 5.

FIG. 4B depicts a bar diagram of the relative expression of STAT1 of FIG. 3 normalized to β-actin expression of FIG. 5.

FIG. 5 shows a Western blotting analysis of the protein expression of PAI-1 (about 50 kDa), P21^{Waf1/Cip1} (about 20 kDa), α-SMA (about 42 kDa) and fibronectin (about 200 kDa) in the renal cortex tissues of rats according to another embodiment of the present invention.

FIG. 6A depicts a bar diagram of the relative expression of PAI-1 normalized to α-tubulin expression of FIG. 5.

FIG. 6B depicts a bar diagram of the relative expression of P21^{Waf1/Cip1} normalized to β-actin expression of FIG. 5.

FIG. 6C depicts a bar diagram of the relative expression of α-SMA normalized to β-actin expression of FIG. 5.

FIG. 6D depicts a bar diagram of the relative expression of fibronectin normalized to β-actin expression of FIG. 5.

### DETAILED DESCRIPTION OF THE PREFERRED EMBODIMENT

Accordingly, the present invention provides a use of probiotic strain GM-263 (ADR-1) with a therapeutically effective amount for treating renal fibrosis in diabetes.

The term “probiotic strain GM-263 (ADR-1)” described herein refers to Lactobacillus reuteri strain GM-263 (ADR-1). L. reuteri strain GM-263 (ADR-1) has been deposited with the China Center for Type Culture Collection (CCTCC), Wuhan University, Wuhan 430072, People's Republic of China under accession number of CCTCC M 209263 on Nov. 13, 2009 under the Budapest Treaty and has CCTCC M 209263 as an internal Patent Deposit Designation and GM-263 (ADR-1) as a Depositor Identification Reference.

The artisan in this art is familiar with the probiotic strain GM-263 (ADR-1) inhabited in a tissue sample obtained by the prior methods or isolated from the gastrointestinal tract (e.g. stomach, intestine or duodenum) of healthy human volunteers, followed by a series of microbiological examinations (e.g. morphological, physiological, genetic characteristics, 16S rDNA sequence analysis, any commercially available product with equivalent function such as API identification system and so on) rather than being recited in detail herein.

In brief, the probiotic strain GM-263 (ADR-1) can be cultured in MRS broth medium (DIFCO®0881) (final pH 6.5±0.2) at 37° C under an anaerobic condition. In another example, the MRS broth of the probiotic strain GM-263 (ADR-1) culture may be streaked onto an agar plate.

In an embodiment, the probiotic strain GM-263 (ADR-1) of the present invention can specifically reduce the concentration of glycated hemoglobin and blood sugar and keep body weight and kidney weight within normal range, which are indicated by a series of in vivo animal immune experiments. Moreover, the probiotic strain GM-263 (ADR-1) of the present invention can also specifically inhibit phosphorylation of JAK2/STAT1 signal transduction pathway and renal fibrosis-related protein expression (e.g. PAI-1, CDKI P21^{Waf1/Cip1}; α-SMA and fibronectin), which are also demonstrated by in vivo animal immune experiments, thereby treating renal fibrosis in diabetes.

It should be clarified that, the term “animal experiments” described herein refers to employ “STZ-induced diabetic rats” to evaluate the effect of the probiotic strain GM-263 (ADR-1) for preventing the renal fibrosis in diabetes, in which those animals are artificially induced by using drugs such as streptozotocin (STZ) to induce diabetes. However, as is understood by a person skilled in the art, the probiotic strain GM-263 (ADR-1) can be treated on any diabetic subject rather than being limited in rats.

It should be supplemented that the probiotic strain GM-263 (ADR-1) (for example, Lactobacillus reuteri strain GM-263 (ADR-1); accession No. CCTCC M 209263) may be live or inactive when it is applied in the composition for treating renal fibrosis in diabetes. In an example, the probiotic strain GM-263 (ADR-1) may be a medical composition, a food additive, a food or its ingredient. In another example, the probiotic strain GM-263 (ADR-1) may be lyophilized, and the probiotic strain GM-263 (ADR-1) may further include other ingredients, for example, glucose, maltodextrin, baby milk, fructo-oligosaccharides, magnesium stearate, yogurt spices, other uncertain remains unseparated therefrom or any combination thereof.

In addition, the probiotic strain GM-263 (ADR-1) (for example, Lactobacillus reuteri strain GM-263 (ADR-1); accession No. CCTCC M 209263) alternatively include other strains so as to produce a composition for treating renal fibrosis in diabetes. In an example, the other strains may include but not be limited in Lactobacillus acidophilus, Lactobacillus plantarum, Bifidobacterium longum, Lactobacillus fermentum, Lactobacillus bulgaricus, Streptococcus themophilus, Lactobacillus cremors, Lactobacillus paracasei subsp. paracasei, Lactobacillus rhamnosus GG or any combination thereof.

In an embodiment, there is only one single step of administrating a therapeutically effective amount of probiotic strain GM-263 (ADR-1) of Lactobacillus reuteri strain GM-263 (ADR-1) (accession No.: CCTCC M 209263) is included in a method for treating renal fibrosis in diabetes, so as to specifically inhibit phosphorylation of JAK2 and STAT1 and to inhibit renal fibrosis-related protein expression (e.g. PAI-1, CDKI P2.1^{Waf1/Cip1}, α-SMA and fibronectin).

Thereinafter, various applications of the probiotic strain GM-263 (ADR-1) will be described in more details referring to several exemplary embodiments below, while not intended to be limiting. Thus, one skilled in the art can easily ascertain the essential characteristics of the present invention and, without departing from the spirit and scope thereof, can make various changes and modifications of the invention to adapt it to various usages and conditions.

### EXAMPLE 1

### Establishment of STZ-Induced Diabetic Rats

1. Preparation of Probiotic Strain GM-263 (ADR-1)

In this EXAMPLE, the probiotic strain GM-263 (ADR-1), Lactobacillus reuteri strain GM-263 (ADR-1; accession No. CCTCC M 209263) may be used in animal experiments, so as to evaluate the effect of the probiotic strain GM-263 (ADR-1) for preventing the renal fibrosis in diabetes.

A partial 16S rDNA sequence of the probiotic strain GM-263 (ADR-1; accession No. CCTCC M 209263) is listed as a sequence of SEQ ID NO.: 1 and analyzed by the Food Industry Research and Development Institute (FIRDI, Hsinchu, Taiwan). The sequence of SEQ ID NO.: 1 includes 560 nucleotides and has a 99% similarity (i.e. sequence identity) compared with 16S rDNA sequence of L. reuteri.

Moreover, the “species name” of the probiotic strain GM-263 (ADR-1; accession No. CCTCC M 209263) can be identified by using API® 50 CHL strip (bioMérieux Inc., France) or any commercially available product with equivalent function, in comparison with a standard bacterial strain such as Lactobacillus reuteri (ATCC 23272).

Results obtained from the probiotic strain GM-263 (ADR-1; accession No. CCTCC M 209263) in the API® 50 CHL strip are shown in TABLE 1.

TABLE 1

| | Probiotic strain | |
|---|---|---|
| | GM-263 (ADR-1) | L. reuteri (ATCC |
| API test | (CCTCC M 209263) | 23272) |
| Glycerol | − | − |
| Erythritol | − | − |
| D-Arabinose | − | − |
| L-Arabinose | + | + |
| D-Ribose | − | + |
| D-Xylose | − | − |
| L-Xylose | − | − |
| D-Adonitol | − | − |
| Methyl-D-Xylopyranoside | − | − |
| D-Galactose | + | + |
| D-Glucose | + | + |
| D-Fructose | − | + |
| D-Mannose | − | − |
| L-Sorbose | − | − |
| L-Rhamnose | − | − |
| Dulcitol | − | − |
| Inositol | − | − |
| D-Mannitol | − | − |
| D-Sorbitol | − | − |
| Methyl-D-Mannopyranoside | − | − |
| Methyl-D-Glucopyranoside | − | − |
| N-AcetylGlucosamin | − | − |
| Amygdalin | − | − |
| Arbutin | − | − |
| Esculin | − | − |
| Salicin | − | − |
| D-Celiobiose | − | − |
| D-Maltose | + | + |
| D-Lactose | − | + |
| D-Melibiose | + | + |
| D-Saccharose | + | + |
| D-Trehalose | − | − |
| Inulin | − | − |
| D-Melezitose | − | − |
| D-Raffinose | + | + |
| Amidon | − | − |
| Glycogen | − | − |
| Xylitol | − | − |
| Gentiobiose | − | − |
| D-Turanose | − | − |
| D-Lyxose | − | − |
| D-Tagatose | − | − |
| D-Fucose | − | − |
| L-Fucose | − | − |
| D-Arabitol | − | − |
| L-Arabitol | − | − |
| Potassium Gluconate | + | + |
| Potassium 2-Ketogluconate | − | − |
| Potassium 5-Ketogluconate | − | − |

| | |
|---|---|
| Matched numbers between the probiotic strain | 46 |
| GM-263 (ADR-1) and L. reuteri (ATCC 23272): | |

Ps. “+” is referred to positive reaction, and “−” is referred to negative reaction

According to the results of 16S rDNA sequence analysis and TABLE 1, the probiotic strain GM-263 (ADR-1) is very similar to L. reuteri (ATCC 23272) in 16S rDNA sequence, metabolism and physiological behaviors. Therefore, the probiotic strain GM-263 (ADR-1) is identified as L. reuteri.

The identified L. reuteri strain GM-263 (ADR-1; accession No. CCTCC M 209263) may be cultured in MRS broth medium (DIFCO®0881) (final pH 6.5±0.2) at 37° C. under an anaerobic condition. Alternatively, the MRS broth of the probiotic strain GM-263 (ADR-1) culture may be streaked onto an agar plate. The L. reuteri strain GM-263 (ADR-1; accession No. CCTCC M 209263) may be further cultured in mass production for the use of subsequent animal experiments.

During animal experiments, L. reuteri strain GM-263 (ADR-1; accession No. CCTCC M 209263) may have a dosage of 1×10⁶ to 1×10¹¹ CFU/g (colony-forming units per gram). L. reuteri strain GM-263 (ADR-1) may be lyophilized, and reuteri strain GM-263 (ADR-1) may further include other ingredients as excipients, for example, glucose, maltodextrin, baby milk, fructo-oligosaccharides, magnesium stearate, yogurt spices, other uncertain remains unseparated therefrom or any combination thereof. For example, the excipients for lyophilization of L. reuteri strain GM-263 (ADR-1; accession No. CCTCC M 209263) may be skim milk, trehalose and a fructooligosaccharide mixture [2:1:1 (w/v)].

2. Establishment of STZ-Induced Diabetes In Rats

In this EXAMPLE, male Sprague-Dawley (S.D.) rats (purchased from BioLASCO Taiwan Co., Ltd., Taipei, Taiwan) at an age of 12 weeks are used to establish STZ-Induced Diabetic rats. All rats are fasted for 18 hours and then diabetes is induced by intraperitoneal injection of streptozotocin (STZ; Sigma-Aldrich Chemical, St. Louis, Mo., U.S.A.) at 50 mg/kg body weight, which is freshly dissolved in 10 mM sodium citrates, pH 4.5. After two days, the induction of diabetes is confirmed by measurement of the blood glucose using the glucose oxidase method, and hyperglycemic rats with blood glucose levels higher than 16 mmol/L are used. Blood glucose is monitored everyday.

Rats were divided into five groups: (i) normal group (Normal, n=6), fed standard chow (contents of 20.60% fat, 56.57% carbohydrate and 22.83% protein); (ii) diabetic group (DM, n=6), fed with standard chow; (iii) diabetic control group, treated with insulin (4-5 U/kg/day) (DM+Ins, n=6), fed with standard chow; (iv) L. reuteri GM-263 (ADR-1) group (GM-263, n=6), fed L. reuteri GM-263 (ADR-1) (1×10⁹ organisms/rat/day) along with standard chow; and (v) GM-263 (ADR-1) diabetic group (DM+GM-263, n=6), fed L. reuteri GM-263 (ADR-1) (1×10⁹ organisms/rat/day) and standard chow. No significant difference Was noted in dietary intake between the groups (25.6±0.77 g/day; p=0.1747). Experimental materials were suspended in 0.5 mL PBS and orally administered twice a day. On study day 28, after streptozotocin treatment, the body weight and the kidney weight of all rats are recorded. And then, all rats are sacrificed and renal cortex of all rats is dissected for further analysis.

Ambient temperature is controlled at 25±1° C., relative humidity at 65±5%. In addition, the rats are maintained on a reverse 12 h light-dark cycle. Rats are provided with standard laboratory chow (LabDiet® Laboratory Rodent Diet #5001, PMI Nutrition International Inc., U.S.A) and water ad libitum). All experimental procedures are approved according to the NIH Guide for the Care and Use of Laboratory Animals.

### EXAMPLE 2

### Evaluation of Probiotic Strain GM-263 (ADR-1) Treatment on Physiological Influence of STZ-Induced Diabetic Rats

### 1. Evaluation of Probiotic Strain GM-263 (ADR-1) Treatment on Glycated Hemoglobin and Blood Glucose in STZ-Induced Diabetic Rats

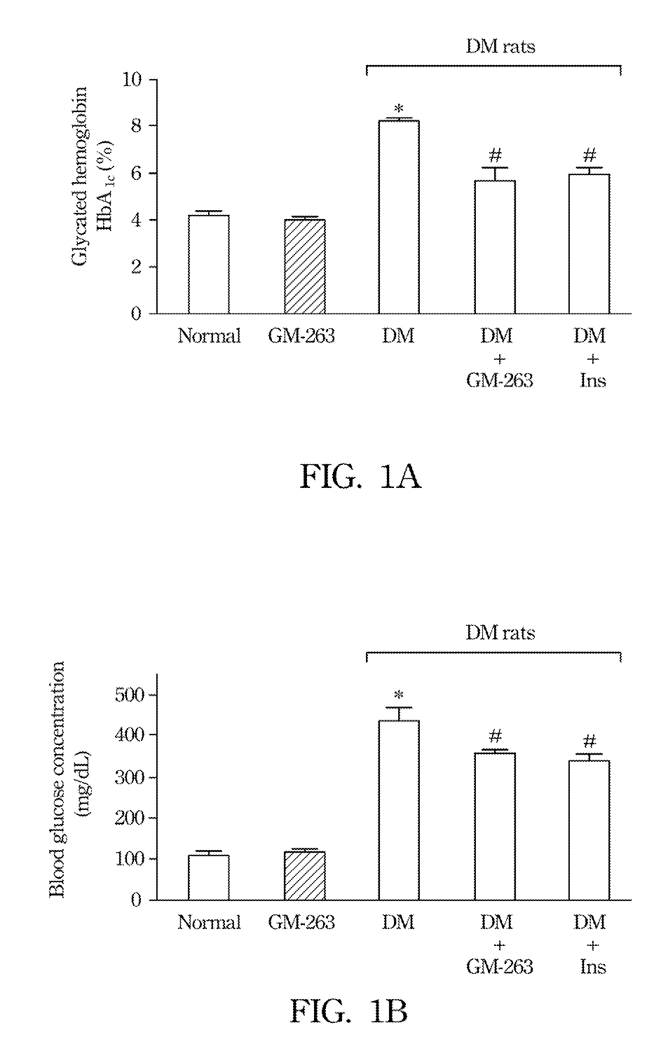

Reference is made to FIGS. 1A and 1B, which show bar diagrams of L. reuteri GM-263 (ADR-1) treatment on glycated hemoglobin (FIG. 1A) and blood glucose (FIG. 1B) in rats. The vertical axis of FIG. 1A is referred to percent (%) of glycated hemoglobin (HbA_{1c}), and the vertical axis of FIG. 1B is referred to percent (%) of blood glucose (mg/dL). The horizontal axis of FIGS. 1A and 1B are referred to the normal group (Normal), the L. reuteri GM-263 (ADR-1) group (GM-263), the diabetic group (DM), the L. reuteri GM-263 (ADR-1) diabetic group (DM+GM-263) and the diabetic control group (DM+Ins), respectively. The symbol * means that the group has p<0.05 in comparison with the normal group (Normal), and the symbol # means that the group has p<0.05 in comparison with the diabetic group (DM).

According to the results of FIGS. 1A and 1B, the DM group obviously increases glycated hemoglobin (more than 7.5%) and blood glucose (more than 350 mg/dL) than the rats of normal group and GM-263 group for 28 days after STZ injection. In normal rats, oral treatment (1×10⁹ organisms/rat/day) of L. reuteri GM-263 maintains both glycated hemoglobin and blood glucose levels similar to those of normal rats without fed L. reuteri GM-263. However, in DM rats, the glycated hemoglobin and blood glucose are significantly decreased by oral treatment of L. reuteri GM-263 and insulin (4-5 U/kg/day) administration. The result shows that the glycated hemoglobin and blood glucose levels of the DM rats can helpfully decreased by oral treatment of the probiotic strain GM-263 (ADR-1) of EXAMPLE 1.

### 2. Evaluation of Probiotic Strain GM-263 (ADR-1) Treatment on Body Weight and Kidney Weight of STZ-Induced Diabetic Rats

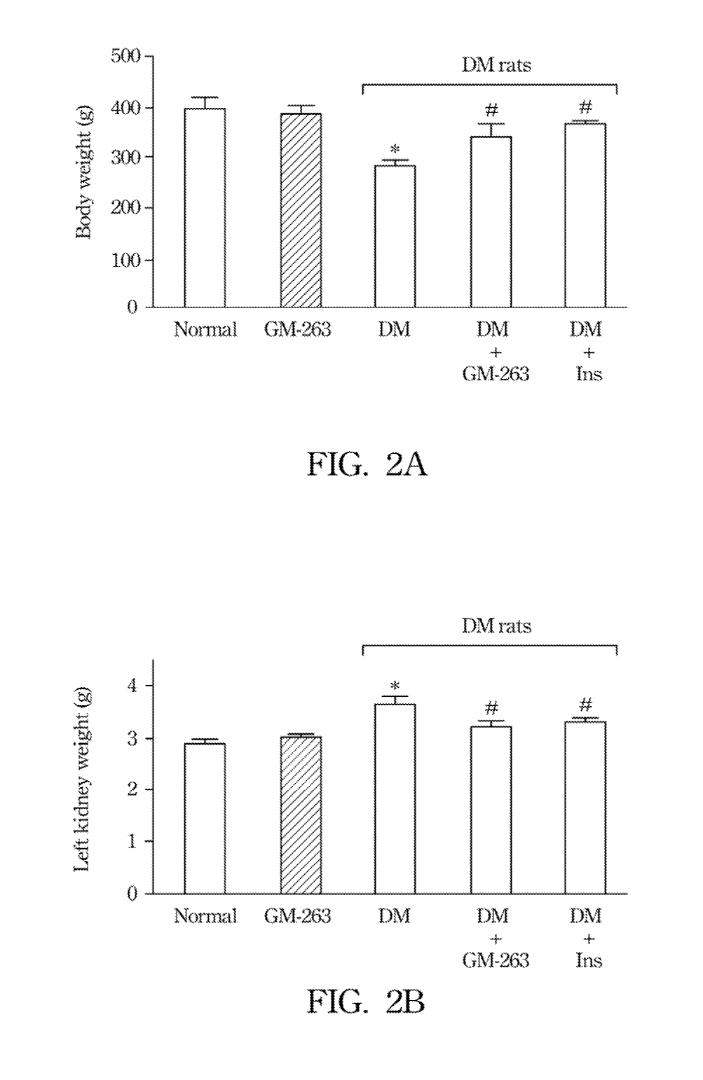

Reference is made to FIGS. 2A and 2B, which show bar diagrams of L. reuteri GM-263 (ADR-1) treatment on body weight (FIG. 2A) and left kidney weight (FIG. 2B) of rats. The vertical axis of FIG. 2A is referred to body weight (g), and the vertical axis of FIG. 2B is referred to left kidney weight (g). The horizontal axis of FIGS. 2A and 2B are referred to the normal group (Normal), the L. reuteri GM-263 (ADR-1) group (GM-263), the diabetic group (DM), the L. reuteri GM-263 (ADR-1) diabetic group (DM+GM-263) and the diabetic control group (DM+Ins), respectively. The symbol * means that the group has p<0.05 in comparison with the normal group (Normal), and the symbol # means that the group has p<0.05 in comparison with the diabetic group (DM).

According to the results of FIGS. 2A and 2B, in normal rats, the body weight and the left kidney weight of the L. reuteri GM-263 (ADR-1) group (GM-263) has no significant difference with those of the normal rats. However, the DM group has lower body weight than the L. reuter GM-263 group and normal group for 28 days after STZ injection (FIG. 2A). In the DM group, kidney weight as a parameter of renal fibrosis is significantly greater than that of the normal group and the L. reuteri GM-263 (ADR-1) group (GM-263) (FIG. 2B).

Interestingly, L. reuteri GM-263 (ADR-1) or insulin; administration slightly increase body weight (FIG. 2A) of DM rats. Moreover, L. reuteri GM-263 (ADR-1) diabetic group (DM+GM-263) or diabetic control group (DM+Ins) have lower kidney weight than diabetic group (DM) rats (FIG. 2B). Therefore, these data suggest that treatment with L. reuteri GM-263 (ADR-1) of EXAMPLE may be an effective strategy for improving renal fibrosis in diabetic subjects.

### EXAMPLE 3

### Evaluation of Probiotic Strain GM-263 (ADR-1) Treatment Involving in Gene Regulation and Cellular Protein Expression in Renal Cortex of STZ-Induced Diabetic Rats

1. Kidney Tissue Extraction

For protein extraction, renal cortex of EXAMPLE 1 is homogenized in 0.1% sodium dodecyl sulfate (SDS) lysis buffer (containing 50 mM Tris-Cl, pH 8.0; 150 mM NaCl; 0.02% sodium azide; 1% NP-40; 0.05% Na₃VO₄; 100 μg/mL phenylmethylsulfonyl fluoride; protease inhibitor cocktail, all purchased from Sigma-Aldrich Chemical, St. Louis, Mo., U.S.A.) in a ratio of about 50 mg tissue/1 mL 0.1% SDS lysis buffer by using a tissue homogenizer at 4° C. or on ice, allowing the mixture to stand for reacting about 10 minutes, for releasing; the proteins in the renal cortex cells.

And then, the homogenate is centrifugated at 12,000 rpm for about 20 minutes. Protein concentration in the supernatant was measured using the Bradford method. Finally, the samples were immediately stored at −70° C. for the subsequent analysis.

2. Western Blotting Assay

The aforementioned homogenate samples are further loaded into 10% sodium dodecyl sulfate-polyacrylamide gel electrophoresis (SDS-PAGE), electrophoresed at 100 V of voltage in a running buffer (including 125 mM Tris-base, 1.25 M Glycine, 1% SDS) for 3 hours and equilibrated for 15 min in 25 mM Tris-HCI, pH 8.3, containing 192 mM glycine and 20% (V/V) methanol. The preparation of SDS-PAGE and related equipments are familiar with the artisan in this art of the present invention rather than being recited in detail herein.

Electrophoresed proteins are transferred to a transfer membrane such as nitrocellulose membrane (Protran™ membrane'; 0.45 μm pore size; Schieicher & Schuell, Kneene, N.H., U.S.A.) with a Western blotting kit, for example, Bio-Rad Scientific Instruments Transfer Unit, at 100 V for 3 hours.

And then, the nitrocellulose membrane is incubated at room temperature for 1 h in blocking buffer containing 5% non-fat milk and a TBS buffer (10 mM Tris-Base, 100 mM NaCl, 0.1% Tween-20, pH 7.4). Primary antibodies are diluted in an antibody-binding buffer overnight at 4° C. The immunoblots are washed three times in a TBS buffer for 10 min during each phase, and then immersed in the secondary antibody solution for 1 h at 37° C. The secondary antibody is diluted 4000-fold. The nitrocellulose membrane is then washed in a TBS buffer three times for 10 min each phase. The immunoblotted proteins are visualized by using an enhanced chemiluminescence ECL Western blotting luminal Reagent (Amersham Corp., Arlington Heights, Ill., U.S.A.) and quantified by a Fujifilm LAS-3000 chemiluminescence detection system (Tokyo, Japan).

The primary antibodies described as the aforementioned may include anti-JAK2, -STAT1, -PAI-1, -P21^{Waf1/Cip1} or -α-SMA antibodies (those are purchased from Santa Cruz Biotechnology, Inc., Santa Cruz, Calif., U.S.A); anti-fibronectin antibody (Chemicon, Temecula, Calif., U.S.A.); anti-phospho-JAK2 and -STAT1 antibodies (Upstate Biotechnology, Inc., Santa Cruz, Calif., U.S.A.); or anti-β-actin (Sigma-Aldrich Chemical, St. Louis, Mo., U.S.A.). The secondary antibody solution described as the aforementioned may contain goat anti-mouse IgG-HRP (horseradish peroxidase) or goat anti-rabbit IgG-HRP and streptavidin-peroxidase conjugates (those are purchased from Amersham Corp., Arlington Heights, Ill., U.S.A.).

The antibody binding buffer described as the aforementioned may be TBS buffer, which contains 10 mM Tris-base, 100 mM NaCl, 0.1% (v/v) Tween-20, pH 7.4.

The intensity of Western blot bands is quantified by densitometric analysis. Results are expressed as the ratio of intensity of the protein of interest to that of β-actin or the indicated protein from the same sample. In addition, each example herein and hereafter is repeated a minimum of three times, and data are expressed as mean±SD. One-way ANOVA with a Tukey-Kramer procedure for multiple comparisons is used to examine the statistical differences between treatments. Differences were considered as significant at p<0.05.

3. Evaluation of Influence of Probiotic Strain GM-263 (ADR-1) Involving in Phosphorylation of JAK2 and STAT1 in STZ-Induced Diabetic Rats

Previous studies have showed that JAK/STATs signal transduction pathways play important roles in the hyperglycemia-induced renal fibrosis in diabetic animals. In this EXAMPLE, influence of the probiotic strain GM-263 (ADR-1) involving in phosphorylation of JAK2 and STAT1 in STZ-induced diabetic rat is evaluated.

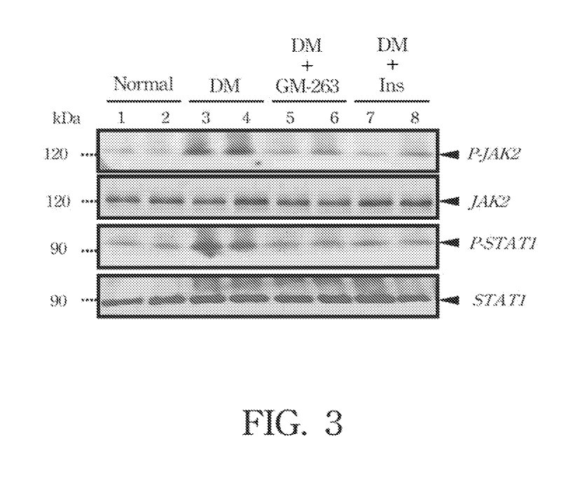

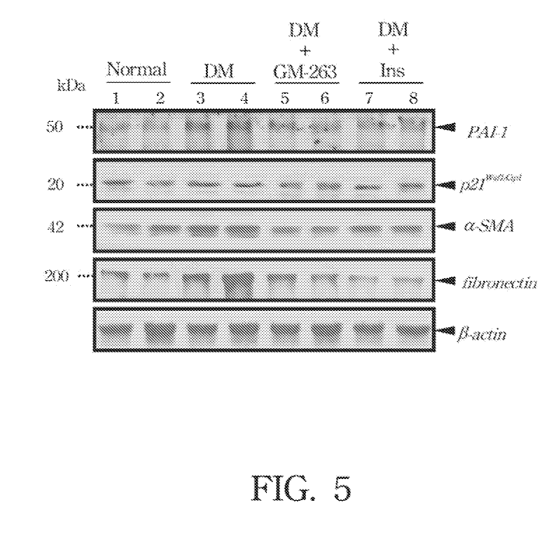

Reference is made to FIG. 3, which shows a Western blotting analysis of renal cortex tissue of rats according to an embodiment of the present invention, in which the lanes 1 to 2 refer to the normal group (Normal), the lanes 3 to 4 refer to the diabetic group (DM), the lanes 5 to 6 refer to the diabetic control group orally administrated with L. reuteri GM-263 (ADR-1) (DM+GM-263), the lanes 7 to 8 refer to the diabetic control group administrated with insulin (DM+Ins). FIG. 3 shows the protein expression of JAK2 (about 120 kDa), STAT1 (about 90 kDa), p-JAK2 (about 120 kDa) and p-STAT1 (about 90 kDa) in the renal cortex tissues of rats. The β-actin amount of FIG. 5 is detected as an internal control for normalizing those protein amounts.

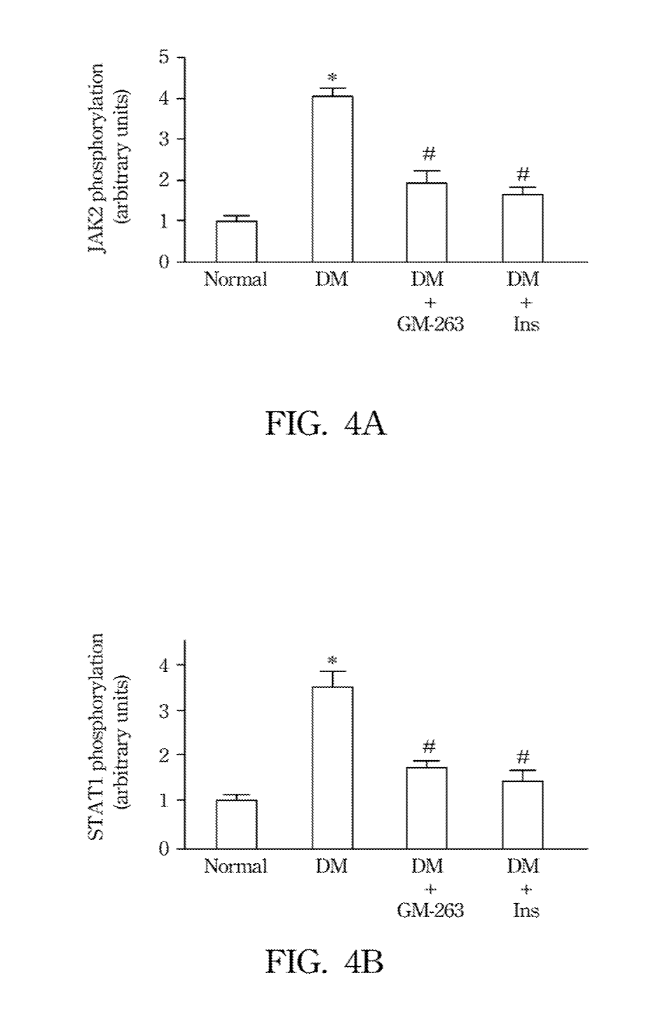

Reference is made to FIG 4A, which depicts a bar diagram of the relative expression of JAK2 of FIG. 3 normalized to β-actin expression of FIG. 5, in which the vertical axis refers to the relative expression of JAK2 normalized to β-actin expression (i.e. relative expression of JAK2/β-actin), and the relative expression of JAK2/β-actin of the normal group is set to 1.0.

Reference is made to FIG. 4B, which depicts a bar diagram of the relative expression of STAT1 of FIG. 3 normalized to β-actin expression of FIG. 5, in which the vertical axis refers to the relative expression of STAT1 normalized to β-actin expression (i.e. relative expression of STAT1/β-actin), and the relative expression of STAT1/β-actin of the normal group is set to 1.0. In FIGS. 4A and 4B, the symbol * means that the group has p<0.05 in comparison with the normal group (Normal), and the symbol # means that the group has p<0.05 in comparison with the diabetic group (DM).

According to the results of FIGS. 3, 4A and 4B, the phosphorylation of JAK2 (lanes 3 to 4 on upper two rows in FIG. 3) and STAT1 (lanes 3 to 4 on lower two rows in FIG. 3) markedly increased in DM group in comparison with the normal group (the lanes 1 to 2 of FIG. 3). The results indicate that JAK2 and STAT1 in renal cortex in DM rats are in highly activated state.

However, L. reuteri GM-263 (ADR-1) (DM+GM-263; lanes 5 to 6 on upper two rows in FIG. 3) or insulin (DM+Ins; lanes 7 to 8 on upper two rows in FIG. 3) administration significantly decreases the phosphorylation of JAK2 and STAT1 in renal cortex in DM rats.

The result indicates that the oral administration of probiotic strain GM-263 (ADR-1) of EXAMPLE 1 specifically inhibits hyperglycemia-enhanced activation of JAK2 and STAT1 in renal cortex in DM rats.

4. Evaluation of Influence of Probiotic Strain GM-263 (ADR-1) Involving in Cellular Protein Expression in STZ-Induced Diabetic Rats

Reference is made to FIG. 5, which shows a Western blotting analysis of renal cortex tissue of rats according to an embodiment of the present invention, in which the lanes 1 to 2 refer to the normal group (Normal), the lanes 3 to 4 refer to the diabetic control group (DM), the lanes 5 to 6 refer to the diabetic control group orally administrated with L. reuteri GM-263 (ADR-1) (DM+GM-263), the lanes 7 to 8 refer to the diabetic control group administrated with insulin (DM+Ins). FIG. 5 shows the protein expression of PAI-1 (about 50 kDa), P21^{Waf1/Cip1} (about 20 kDa), α-SMA (about 42 kDa) and fibronectin (about 200 kDa) in the renal cortex tissues of die rats according to another embodiment of the present invention. The β-actin amount is detected as an internal control for normalizing those protein amounts.

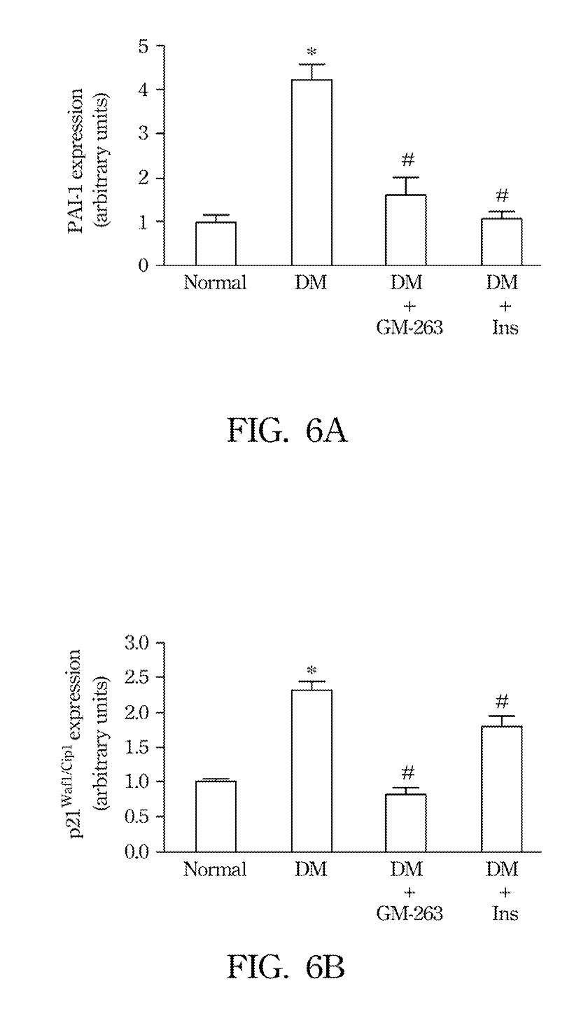

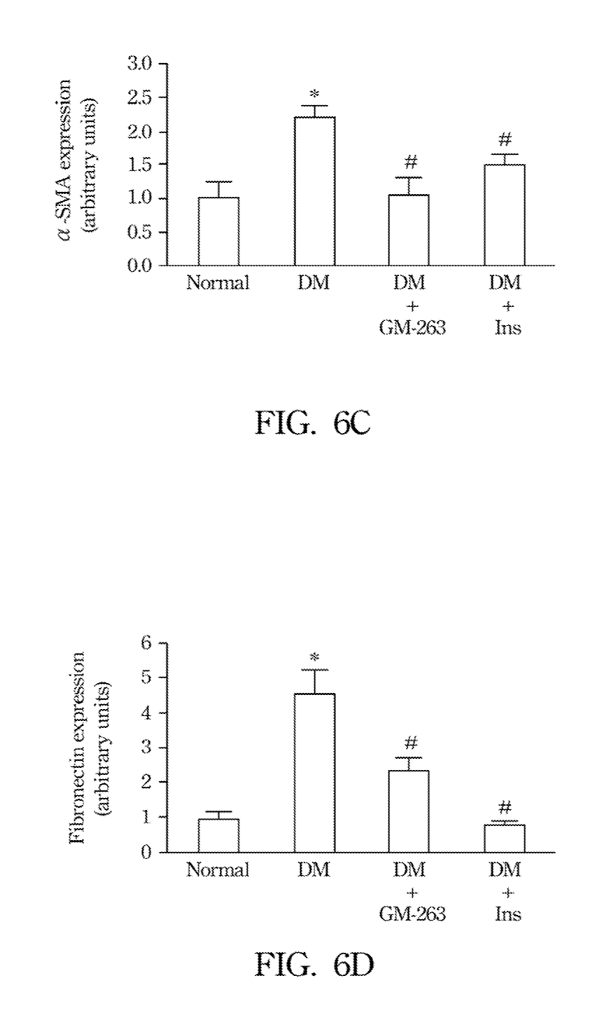

Reference is made to FIG. 6A, which depicts a bar diagram of the relative expression of PAI-1 normalized to p-actin expression of FIG. 5, in which the vertical axis refers to the relative expression of PAI-1 normalized to β-actin expression (i.e. relative expression of PAI-1/β-actin), and the relative expression of PAI-1/β-actin of the normal group is set to 1.0.

Reference is made to FIG. 6B, which depicts a bar diagram of the relative expression of P21^{Waf1/Cip1} normalized to β-actin expression of FIG. 5, in which the vertical axis refers to the relative expression of P²¹^{Waf1/Cip1} normalized to β-actin expression (i.e. relative expression of P21^{Waf1/Cip1}/β-actin), and the relative expression of P21^{Waf1/Cip1}/β-actin of the normal group is set to 1.0.

Reference is made to FIG. 6C, which depicts a bar diagram of the relative expression of α-SMA normalized to β-actin expression of FIG. 5, in which the vertical axis refers to the relative expression of α-SMA normalized to β-actin expression (i.e. relative expression of α-SMA/β-actin), and the relative expression of α-SMA/β-actin of the normal group is set to 1.0.

Reference is made to FIG. 6D, which depicts a bar diagram of the relative expression of fibronectin normalized to β-actin expression of FIG. 5, in which the vertical axis refers to the relative expression of fibronectin normalized to β-actin expression (i.e. relative expression of fibronectin/β-actin), and the relative expression of fibronectin/β-actin of the normal group is set to 1.0.

According to the results of FIGS. 5 and 6A to 6D, the phosphorylation of PAI-1 (lanes 3 to 4 on the first upper row in FIG. 5), P21^{Waf1/Cip1} (lanes 3 to 4 on the second upper row in FIG. 5), α-SMA (lanes 3 to 4 on the third upper row in FIG. 5) and fibronectin (lanes 3 to 4 on the fourth upper row in FIG. 5) markedly increased in renal cortex in DM group in comparison with the normal control group (the lanes 1 to 2 of FIG. 5). The results indicate that PAI-1, P21^{Waf1/Cip1}, α-SMA and fibronectin in renal cortex in DM rats are in highly activated state, leading renal fibrosis in diabetic rats.

However, L. reuteri GM-263 (ADR-1) (DM+GM-263; lanes 5 to 6 in FIG. 5) or insulin (DM+Ins; lanes 7 to 8 in FIG. 3) administration significantly decreases the phosphorylation of PAI-1, P21^{Waf1/Cip1}, α-SMA and fibronectin in renal cortex in DM rats.

The result indicates that, the oral administration of probiotic strain GM-263 (ADR-1) of EXAMPLE 1 specifically inhibits hyperglycemia-enhanced expression of PAI-1, P21^{Waf1/Cip1}, α-SMA and fibronectin in renal cortex in DM rats, thereby protecting STZ-induced diabetic rats from hyperglycemia-enhanced renal fibrosis.

In summary, the present invention is evidenced that the probiotic strain GM-263 (ADR-1) (L. reuteri strain GM-263 (ADR-1); accession No. CCTCC M 209263) of the present invention can be applied in the treatment of renal fibrosis in diabetes, and the probiotic strain GM-263 (ADR-1) is potentially involved in the mechanism of the specific inhibition of the phosphorylation of JAK2/STAT1 signal transduction pathway, and the specific inhibition of protein expression of PAI-1, P21^{Waf1/Cip1}, α-SMA and fibronectin, so as to treat renal fibrosis in diabetes, thereby exploiting other applications of the probiotic strains.

However, it is necessarily supplemented that, specific strains, specific analysis methods, specific animal models, specific reaction conditions, specific immunization ways, specific materials or specific apparatuses are employed as exemplary embodiments for clarifying the use of the probiotic strain GM-263 (ADR-1) for treating renal fibrosis in diabetes of the present invention. However, as is understood by a person skilled in the art, other strains, other analysis methods, other animal models, other reaction conditions, other immunization ways, other comparable materials;or apparatuses can be also employed in the composition for treating renal fibrosis in diabetes of the present invention, rather than limiting to thereto. In addition, when the probiotic strain GM-263 (ADR-1) is applied in a composition of a medical composition, a food additive, a food or its ingredient, the probiotic strain GM-263 (ADR-1) is live, inactivate or lyophilized. Moreover, the probiotic strain GM-263 (ADR-1) may further include other ingredients, for example, glucose, maltodextrin, baby milk, fructo-oligosaccharides, magnesium stearate, yogurt spices, other uncertain remains unseparated therefrom or any combination thereof.

According to the embodiments of the present invention, the aforementioned probiotic strain GM-263 (ADR-1) for treating renal fibrosis in diabetes, the probiotic strain GM-263 (ADR-1) can reduces the concentration of glycated hemoglobin and blood sugar and keeping the body weight and the kidney weight within normal range, as well as specifically inhibiting phosphorylation of JAK2/STAT1 signal transduction pathway and renal fibrosis-related protein (e.g. PAI-1, P21^{Waf1/Cip1}, α-SMA and fibronectin) expression, so as to effectively treat renal fibrosis in diabetes, thereby developing other applications of the probiotic strain GM-263 (ADR-1).

As is understood by a person skilled in the art, the foregoing embodiments of the present invention are illustrated of the present invention rather than limiting of the present invention. It is intended to cover various modifications and similar arrangements included within the spirit and scope of the appended claims. Therefore, die scope of which should be accorded the broadest interpretation so as to encompass all such modifications and similar structure.

What is claimed is:

1. A composition for treating renal fibrosis in diabetes which comprises a therapeutically effective amount of a probiotic strain GM-263 (ADR-1) of Lactobacillus reuteri strain GM-263 (ADR-1) (deposited at the China Center for Type Culture Collection of Wuhan University in China under accession No.: CCTCC M 209263).

2. The composition according to claim 1, wherein the probiotic strain GM-263 (ADR-1) is live or inactivate.

3. The composition according to claim 1, wherein probiotic strain GM-263 (ADR-1) specifically inhibits phosphorylation of Janus kinase 2 (JAK2) and signal transducer and activator of transcription 1 (STAT1).

4. The composition according to claim 1, wherein the probiotic strain GM-263 (ADR-1) specifically inhibits protein expression of plasminogen activator inhibitor (PAI-1), cyclin-dependent kinase inhibitor (CDKI) P21^{Waf1/Cip1}, smooth muscle α-actin (α-SMA) or fibronectin.

5. The composition according to claim 1, wherein the composition is a medical composition, a food additive, a food or its ingredient.

6. The composition according to claim 1, further comprising at least one other strain, wherein the at least one other strain is selected from the group consisting of Lactobacillus acidophilus, Lactobacillus plantarum, Bifidobacterium longum, Lactobacillus fermentum, Lactobacillus bulgaricus, Streptococcus themophilus, Lactobacillus cremors, Lactobacillus paracasei subsp. paracasei, Lactobacillus rhamnosus GG and any combination thereof.

7. A method for treating renal fibrosis in diabetes, comprising:
administrating a therapeutically effective amount of probiotic strain GM-263 (ADR-1) of Lactobacillus reuteri strain GM-263 (ADR-1) (deposited at the China Center for Type Culture Collection of Wuhan University in China under accession No.: CCTCC M 209263) to specifically inhibit phosphorylation of JAK2 and STAT1.

8. The method according to claim 7, wherein the probiotic strain GM-263 (ADR-1) specifically inhibits protein expression of PAI-1, CDKI P21^{Waf1/Cip1}, α-SMA or fibronectin.

9. The method according to claim 7, wherein the probiotic strain GM-263 (ADR-1) is live or inactive.

10. The method according to claim 7, wherein the probiotic strain GM-263 (ADR-1) is further mixed with at least one other strain, and the at least one other strain is selected from the group consisting of Lactobacillus acidophilus, Lactobacillus plantarum, Bifidobacterium longum, Lactobacillus fermentum, Lactobacillus bulgaricus, Streptococcus thermophilus, Lactobacillus cremors, Lactobacillus paracasei subsp. paracasei, Lactobacillus rhamnosus GG and any combination thereof.


SEQUENCE LISTING (1 sequence)

SEQ ID NO: 1 - DNA, 560 bp, Lactobacillus reuteri GM-263 (ADR-1) 16S rDNA
gatgaacgccggcggtgtgcctaatacatgcaagtcgtacgcactggcccaactgattgatggtgcttgcacctgattgacgatggatcaccagtgagtggcggacgggtgagtaacacgtaggtaacctgccccggagcgggggataacatttggaaacagatgctaataccgcataacaacaaaagccacatggcttttgtttgaaagatggctttggctatcactctgggatggacctgcggtgcattagctagttggtaaggtaacggcttaccaaggcgatgatgcatagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttggagaagaacgtgcgtgagagtaactgttcacgcagtgacggtatccaaccagaaagtcacggctaactacgtgccagcagccgcggtaatacgtaggtggcaagcgttat